(19) United States
(12) Patent Application Publication
(10) Pub. No.: US 2022/0344052 A1
(43) Pub. Date: Oct. 27, 2022

(54) INFORMATION PROVISION DEVICE AND METHOD FOR GENERATING MIRNA IMPORTANCE TABLE

(71) Applicant: KEWPIE CORPORATION, Tokyo (JP)

(72) Inventors: Kurataka OTSUKA, Tokyo (JP); Yoshinori KONO, Tokyo (JP); Hideto YOSHIDA, Tokyo (JP); Atsushi MIYATA, Tokyo (JP); Yuko FUNABASHI, Tokyo (JP)

(21) Appl. No.: 17/608,105

(22) PCT Filed: May 28, 2020
(86) PCT No.: PCT/JP2020/021072

§ 371 Date: Nov. 1, 2021

(30) Foreign Application Priority Data
May 8, 2019 (JP) 2019-088334

(51) Int. Cl. G16H 50/30; G16H 10/40; G16B 40/20; G16B 30/00; G16B 20/00

(52) U.S. Cl. CPC G16H 50/30; G16H 10/40; G16B 40/20; G16B 30/00; G16B 20/00

(57) ABSTRACT

An information provision device includes a miRNA divergence value calculation unit 12 that calculates miRNA divergence values from each of the miRNA measurement values in a user miRNA profile and each of the corresponding optimum miRNA values in an optimum miRNA profile, an individual miRNA score calculation unit 13 that calculates individual miRNA scores from each of miRNA importance values of a disease as an object of score calculation listed in a miRNA importance table and each of the miRNA divergence values, and a determination result data generation unit 15 that generates and outputs determination result data based on the individual miRNA scores.

### TECHNICAL FIELD

The present invention relates to an information provision device and a method for generating a miRNA importance table. Priority is claimed on Japanese Patent Application No. 2019-088334 filed on May 8, 2019, the content of which is incorporated herein by reference.

### BACKGROUND ART

Conventionally, a technique of associating the state of human microRNA (miRNA) to the presence of cancer is known (for example, see “Patent Document 1”). Patent Document 1 discloses a technique of associating the expression ratio of miRNA pairs to the presence of lung cancer.

### CITATION LIST

### Patent Document

### [Patent Document 1]

Published Japanese Translation No. 2017 502699 of the PCT International Publication

### SUMMARY OF INVENTION

### Technical Problem

However, using the state of miRNA, the aforementioned conventional technique can contribute to the detection of cancer but cannot contribute to the prevention of cancer.

In recent years, it has been revealed that the imbalance of a miRNA profile is related to the risk of contracting a disease such as cancer. The miRNA expression level can be controlled by the improvement of life habits such as eating habits. Therefore, in a case where a user can ascertain the imbalance of the miRNA expression level (imbalance of a miRNA profile), this will contribute to working out remedies. However, it is difficult for the average user to understand the miRNA profile because it is a complicated profile consisting of approximately 2,500 types of miRNAs. In addition, because miRNA relating to the risk of contracting a disease (morbid risk of a disease) differs according to the type of disease, the imbalance of the miRNA profile cannot be uniformly evaluated.

The present invention has been made in consideration of such circumstances, and an object thereof is to provide an information provision device and a method for generating a miRNA importance table which can concisely present the state of balance of a user's miRNA profile for each type of disease.

### Solution to Problem

The present invention for achieving the above object is realized by the following configuration.

(1) An aspect of the present invention is an information provision device, including a miRNA profile input unit that receives a user miRNA profile constituted of miRNA measurement values of a user, an optimum miRNA profile storage unit that stores an optimum miRNA profile constituted of optimum miRNA values based on a risk of contracting a disease, a miRNA divergence value calculation unit that calculates a miRNA divergence value from each of the miRNA measurement values in the user miRNA profile and each of the corresponding optimum miRNA values in the optimum miRNA profile, the miRNA divergence value showing a divergence between the miRNA measurement value and the optimum miRNA value, a miRNA importance table storage unit that stores, by a type of disease, a miRNA importance table storing miRNA importance values corresponding to each type of disease, an individual miRNA score calculation unit that calculates individual miRNA scores from each of the miRNA importance values of a disease as an object of score calculation listed in the miRNA importance table and each of the miRNA divergence values, and a determination result data generation unit that generates and outputs determination result data based on the individual miRNA scores.

(2) An aspect of the present invention is an information provision device, including a miRNA profile input unit that receives a user miRNA profile constituted of miRNA measurement values of a user, an integrated importance table storage unit that stores an integrated importance table storing integrated miRNA importance values for each type of disease, the integrated miRNA importance values each determined by adding a miRNA importance corresponding to each type of disease to each optimum miRNA value based on a risk of contracting a disease, individual miRNA scores calculation unit that calculates individual miRNA scores from each of the miRNA measurement values in the user miRNA profile and each of the integrated miRNA importance values of a disease as an object of score calculation listed in the integrated importance table, and a determination result data generation unit that generates and outputs determination result data based on the individual miRNA score.

(3) An aspect of the present invention is the information provision device in (1) or (2), further including a miRNA score calculation unit that calculates a miRNA score by combining the individual miRNA scores.

(4) An aspect of the present invention is the information provision device in any of (1) to (3), further including an input unit for specifying a disease as an object of score calculation that receives an input of data for specifying a disease as an object of score calculation showing a disease as an object of score calculation.

(5) An aspect of the present invention is a method for generating a miRNA importance table, including a miRNA disease information acquisition step of acquiring miRNA disease information showing miRNA suppressing or promoting a disease for each type of disease, a miRNA importance calculation step of calculating miRNA importance values corresponding to each type of disease based on the miRNA disease information, and a miRNA importance storage step of storing each of the miRNA importance values calculated for each type of disease in a miRNA importance table.

(6) An aspect of the present invention is a method for generating a miRNA importance table, including a patient miRNA profile acquisition step of acquiring a patient miRNA profile constituted of miRNA measurement values of a patient for each type of disease, an optimum miRNA profile acquisition step of acquiring an optimum miRNA profile constituted of optimum miRNA values based on a risk of contracting a disease, a miRNA importance calculation step of comparing the patient miRNA profile acquired for each type of disease with the optimum miRNA profile and calculating miRNA importance values corresponding to each type of disease based on results of the comparison, and a miRNA importance storage step of storing each of the miRNA importance values calculated for each type of disease in a miRNA importance table.

(7) An aspect of the present invention is a method for generating a miRNA importance table, including a high-risk miRNA profile acquisition step of acquiring a high-risk miRNA profile for each type of disease, the high-risk miRNA profile constituted of miRNA measurement values of an information provider who is above a certain level of risk of contracting a disease but has not yet contracted the disease, an optimum miRNA profile acquisition step of acquiring an optimum miRNA profile constituted of optimum miRNA values based on a risk of contracting a disease, a miRNA importance calculation step of comparing the high-risk miRNA profile acquired for each type of disease with the optimum miRNA profile and calculating miRNA importance values corresponding to each type of disease based on a result of the comparison, and a miRNA importance storage step of storing each of the miRNA importance values calculated for each type of disease.

(8) An aspect of the present invention is the method for generating a miRNA importance table in any one of (5) to (7), further including an information acquisition step of acquiring miRNA attributes, biochemical data, and a miRNA profile of an information provider, in which miRNA importance values corresponding to each type of disease are calculated by additionally using the acquired miRNA attributes, biochemical data, and miRNA profile of the information provider.

### Advantageous Effects of Invention

According to the present invention, it is possible to provide an information provision device and a method for generating a miRNA importance table which can concisely present the state of balance of a user's miRNA profile for each type of disease.

### BRIEF DESCRIPTION OF DRAWINGS

FIG. 1 is a block diagram showing an example of a configuration of an information provision device according to an embodiment of the present invention.

FIG. 2 is a diagram showing an example of a configuration of a user miRNA profile according to an embodiment of the present invention.

FIG. 3 is a diagram showing an example of a configuration of an optimum miRNA profile according to an embodiment of the present invention.

FIG. 4 is a diagram showing an example of a configuration of a miRNA importance table according to an embodiment of the present invention.

FIG. 5 is a flowchart showing an example of an information provision method according to an embodiment of the present invention.

FIG. 6 is a flowchart showing Example 1 of a method for generating a miRNA importance table according to an embodiment of the present invention.

FIG. 7 is a flowchart showing Example 2 of a method for generating a miRNA importance table according to an embodiment of the present invention.

FIG. 8 is a flowchart showing Example 3 of a method for generating a miRNA importance table according to an embodiment of the present invention.

FIG. 9 is a block diagram showing an example of a configuration of an information provision device according to an embodiment of the present invention.

FIG. 10 is a diagram showing an example of a configuration of an integrated importance table according to an embodiment of the present invention.

### DESCRIPTION OF EMBODIMENTS

Hereinafter, modes (hereinafter, called “embodiments”) for carrying out the present invention will be specifically described with reference to the accompanying drawings.

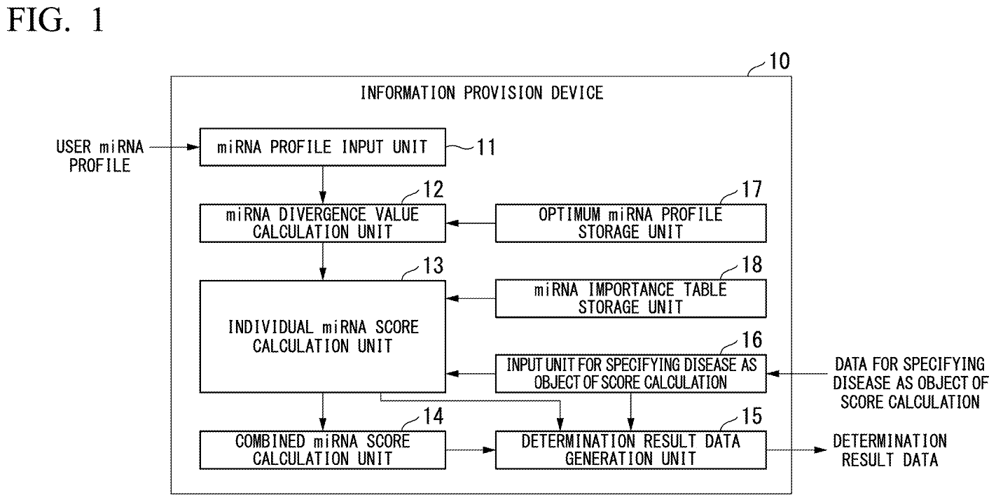

FIG. 1 is a block diagram showing an example of a configuration of an information provision device according to an embodiment. In FIG. 1, an information provision device 10 includes a miRNA profile input unit 11, a miRNA divergence value calculation unit 12, an individual miRNA score calculation unit 13, a combined miRNA score calculation unit 14, a determination result data generation unit 15, an input unit 16 for specifying a disease as an object of score calculation, an optimum miRNA profile storage unit 17, and a miRNA importance table storage unit 18.

The information provision device 10 is configured with a memory, CPU (Central Processing Unit), and the like. The CPU in the information provision device 10 executes computer programs, which enables each part of the information provision device 10 to perform its function. The information provision device 10 may be configured with a general-purpose computer device or configured with a dedicated hardware device.

As peripheral devices, an input device, a display device, and the like may be connected to the information provision device 10. The input device refers to an input device such as a keyboard or a mouse. The display device refers to CRT (Cathode Ray Tube), a liquid crystal display device, or the like. Furthermore, the peripheral devices may be connected to the information provision device 10 directly or through a communication channel.

The information provision device 10 may transmit and receive data to and from an external device such as a user's terminal device through a communication channel. In addition to the user's terminal device, for example, servers of information-providing institutions such as medical institutions may also be used as external devices. The user's terminal device may be, for example, a mobile terminal device, such as a smartphone or a tablet-type computer (tablet PC), or a stationary terminal device (for example, a stationary personal computer or the like).

The information provision device 10 may be realized in a manner in which a server computer connected to a communication network such as the Internet executes computer programs for realizing the function of the information provision device 10. In addition, some of the functions of the information provision device 10 may be provided in a terminal device as a script operating on the browser of the terminal device or as an application installed in the terminal device, and all the functions of the information provision device 10 may be realized by the cooperation of the terminal device having some of the functions of the information provision device 10 and a server computer having the rest of the functions of the information provision device 10.

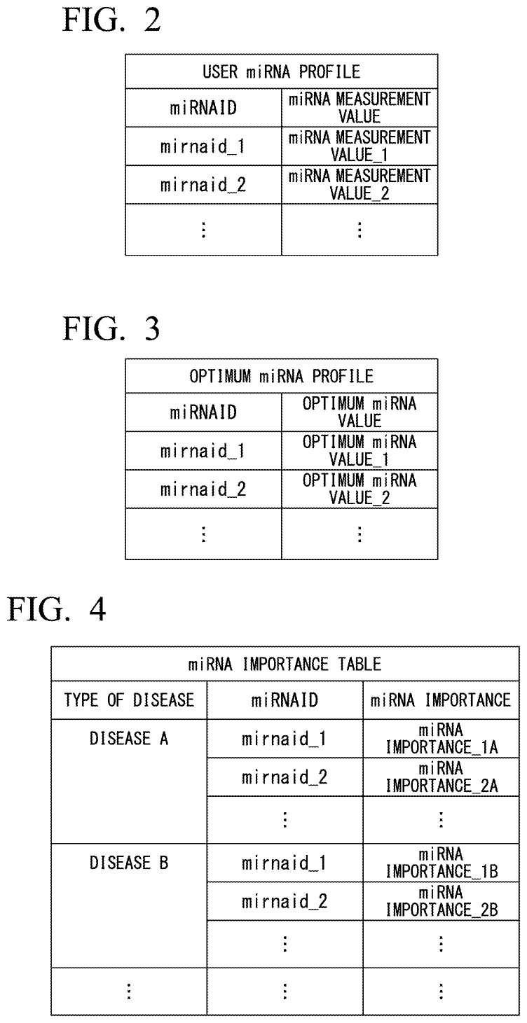

The miRNA profile input unit 11 receives a user miRNA profile input into the information provision device 10. The user miRNA profile is data of a miRNA profile constituted of a group of levels of each miRNA measured (miRNA measurement values) for the user. FIG. 2 shows an example of a configuration of a user miRNA profile. In the user miRNA profile in FIG. 2, miRNA identifiers (miRNAID) and miRNA measurement values constituting the user miRNA profile are stored by being associated with each other.

The miRNA measurement values constituting the user miRNA profile may be miRNA measurement values of all miRNAs, or miRNA measurement values of only the specific miRNAs which are related to a risk of contracting a disease that the information provision device 10 can handle as an object of score calculation. The miRNA measurement value may be any of the miRNA concentration, the number of moles of miRNA, the fluorescence intensity of miRNA, and relative values corrected with other indices. The miRNA measurement value may be expressed using a logarithm method.

The optimum miRNA profile storage unit 17 stores an optimum miRNA profile.

The optimum miRNA profile is data of a miRNA profile constituted of a group of optimum values of each miRNA (optimum miRNA values) based on a risk of contracting a disease. FIG. 3 shows an example of a configuration of an optimum miRNA profile. In the optimum miRNA profile in FIG. 3, miRNAIDs of each miRNA and optimum miRNA values constituting the optimum miRNA profile are stored by being associated with each other.

The optimum miRNA values constituting the optimum miRNA profile may be optimum miRNA values of all miRNAs, or may be optimum miRNA values of only the specific miRNAs which are related to a risk of contracting a disease that the information provision device 10 can handle as an object of score calculation. The optimum miRNA value is the optimum value of miRNA based on a risk of contracting a disease, and may be any of the miRNA concentration, the number of moles of miRNA, the fluorescence intensity of miRNA, or relative values corrected using other indices. The optimum miRNA value may be expressed using a logarithm method. The method for generating the optimum miRNA profile will be described later.

From the viewpoint of miRNA divergence value calculation processing in the miRNA divergence value calculation unit 12 which will be described later, it is preferable that the miRNA measurement values in the user miRNA profile and the optimum miRNA values in the optimum miRNA profile be expressed by the same method.

The miRNA divergence value calculation unit 12 calculates a divergence value of each miRNA (miRNA divergence value) from each of the miRNA measurement values in the user miRNA profile and each of the corresponding optimum miRNA values in the optimum miRNA profile. The miRNA divergence value is a value showing a divergence between a miRNA measurement value and an optimum miRNA value for one miRNA. The miRNA divergence value can be calculated using methods such as performing subtraction or division between the miRNA measurement value and the optimum miRNA value or performing subtraction or division between the logarithms of the miRNA measurement value and the optimum miRNA value. For example, a difference obtained as a result of performing subtraction between the miRNA measurement value and the optimum miRNA value may be adopted as the miRNA divergence value.

The miRNA importance table storage unit 18 stores a miRNA importance table for each type of disease. The miRNA importance table is tabular data that stores the importance of each miRNA (miRNA importance) corresponding to the type of disease. FIG. 4 shows an example of a configuration of a miRNA importance table. In FIG. 4, a miRNA importance table is provided for each type of disease. In the miRNA importance table, miRNAID of each miRNA and miRNA importance thereof constituting the miRNA importance table are stored by being associated with each other. The miRNA importance constituting the miRNA importance table may be miRNA importance of all miRNAs, or may be miRNA importance of only the specific miRNAs related to a risk of contracting a disease targeted by the miRNA importance table. The method for generating the miRNA importance table will be described later.

The individual miRNA score calculation unit 13 calculates an individual score of each miRNA (individual miRNA score) from each of the miRNA importance values of a disease as an object of score calculation listed in the miRNA importance table and each of the miRNA divergence values calculated by the miRNA divergence value calculation unit 12. For example, the product obtained as a result of multiplying the miRNA divergence value by the miRNA importance may be calculated as an individual miRNA score. The input unit 16 for specifying a disease as an object of score calculation notifies the individual miRNA score calculation unit 13 of the disease as an object of score calculation.

The data for specifying a disease as an object of score calculation is input into the input unit 16 for specifying a disease as an object of score calculation. The data for specifying a disease as an object of score calculation is data showing a disease as an object of score calculation. The input unit 16 for specifying a disease as an object of score calculation receives the input of the data for specifying a disease as an object of score calculation. The input unit 16 for specifying a disease as an object of score calculation notifies the individual miRNA score calculation unit 13 and the determination result data generation unit 15 of a disease as an object of score calculation shown in the data for specifying a disease as an object of score calculation. Examples of the data for specifying a disease as an object of score calculation include data for specifying a disease as an object of score calculation that a user transmits to the information provision device 10 by using a terminal device. In this case, the input unit 16 for specifying a disease as an object of score calculation receives the data for specifying a disease as an object of score calculation from the user's terminal device, and notifies the individual miRNA score calculation unit 13 of the disease as an object of score calculation shown in the received data for specifying a disease as an object of score calculation.

As the disease as an object of score calculation, all the diseases that the information provision device 10 can handle as an object as score calculation may be pre-set in the information provision device 10 as a disease as an object of score calculation. In this case, the information provision device 10 may not include the input unit 16 for specifying a disease as an object of score calculation. In a case where all the diseases that the information provision device 10 can handle as an object of score calculation are pre-set in the information provision device 10 as a disease as an object of score calculation, the individual miRNA score calculation unit 13 calculates individual miRNA scores for every single type of disease listed in the miRNA importance table.

The combined miRNA score calculation unit 14 calculates a combined miRNA score by combining the individual miRNA scores of a disease as an object of score calculation calculated by the individual miRNA score calculation unit 13. The combined miRNA score is calculated for each type of disease. In a case where the number of diseases as objects of score calculation is more than one, the combined miRNA score is calculated by combining the individual miRNA scores of each of the diseases as objects of score calculation.

The determination result data generation unit 15 generates and outputs determination result data. The determination result data is the combined miRNA score, the individual miRNA score, data showing the individual miRNA score in a diagram, or the like. The combined miRNA score is transmitted to the determination result data generation unit 15 from the combined miRNA score calculation unit 14. The individual miRNA score is transmitted to the determination result data generation unit 15 from the individual miRNA score calculation unit 13. As the individual miRNA score to be output as the determination result data, a plurality of individual miRNA scores strongly associated with the disease as an object of score calculation are selected.

As data showing the individual miRNA scores in a diagram, for example, a radar chart is generated. The individual miRNA scores used in the radar chart are a plurality of individual miRNA scores selected as being strongly associated with the disease as an object of score calculation. The input unit 16 for specifying a disease as an object of score calculation notifies the combined miRNA score calculation unit 14 of the disease as an object of score calculation. The determination result data may be one or two or more out of the combined miRNA score, the individual miRNA score, and data showing the individual miRNA score in a diagram, such as a radar chart.

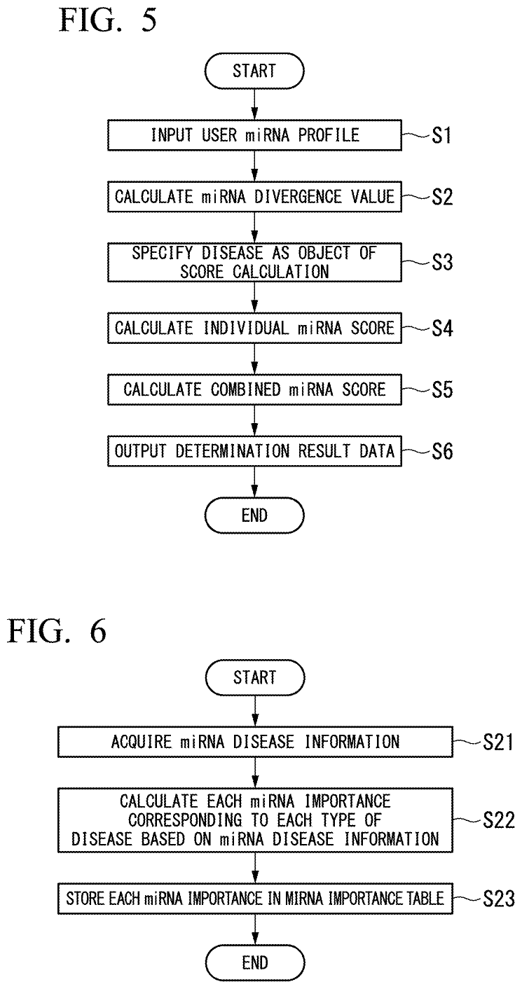

Next, the operation of the information provision device 10 shown in FIG. 1 will be described with reference to FIG. 5. FIG. 5 is a flowchart showing an example of an information provision method according to the present embodiment.

(Step S1) A user miRNA profile is input into the information provision device 10. The user miRNA profile may be transmitted to the information provision device 10 from the user's terminal device or from an external device other than the user's terminal device. The external device other than the user's terminal device may be, for example, servers of information-providing institutions such as medical institutions.

The miRNA profile input unit 11 receives the user miRNA profile input into the information provision device 10. The miRNA profile input unit 11 transmits the user miRNA profile to the miRNA divergence value calculation unit 12.

(Step S2) The miRNA divergence value calculation unit 12 calculates miRNA divergence values from each of the miRNA measurement values in the user miRNA profile received from the miRNA profile input unit 11 and each of the corresponding optimum miRNA values in the optimum miRNA profile stored in the optimum miRNA profile storage unit 17. The miRNA divergence value calculation unit 12 transmits the calculated miRNA divergence values to the individual miRNA score calculation unit 13.

(Step S3) The input unit 16 for specifying a disease as an object of score calculation notifies the individual miRNA score calculation unit 13 and the determination result data generation unit 15 of a disease as an object of score calculation shown in the data for specifying a disease as an object of score calculation. In Step S3, for example, the disease as an object of score calculation is “disease A”. Therefore, in this step, the input unit 16 for specifying a disease as an object of score calculation notifies the individual miRNA score calculation unit 13 and the determination result data generation unit 15 of a disease as an object of score calculation, which is “disease A”.

(Step S4) The individual miRNA score calculation unit 13 calculates individual miRNA scores from each of the miRNA importance values of the disease A listed in the miRNA importance table stored in the miRNA importance table storage unit 18 and each of the miRNA divergence values received from the miRNA divergence value calculation unit 12. The individual miRNA score calculation unit 13 transmits the calculated individual miRNA scores of the disease A to the combined miRNA score calculation unit 14.

(Step S5) The combined miRNA score calculation unit 14 calculates a combined miRNA score by combining the individual miRNA scores of the disease A received from the individual miRNA score calculation unit 13. The combined miRNA score calculation unit 14 transmits the calculated combined miRNA score of the disease A to the determination result data generation unit 15.

(Step S6) The determination result data generation unit 15 generates and outputs determination result data of the disease A. The determination result data is the combined miRNA score, the individual miRNA score, data showing the individual miRNA score in a diagram, such as a radar chart, or the like. The determination result data may be one or two or more out of the combined miRNA score, the individual miRNA score, and data showing the individual miRNA score in a diagram, such as a radar chart. The destination of the determination result data to be output is set in advance. The destination of the determination result data to be output may be a user's terminal device or an external device other than the user's terminal device. The external device other than the user's terminal device may be, for example, servers of information-providing institutions such as medical institutions.

### Method for Generating Optimum miRNA Profile

Next, a method for generating an optimum miRNA profile according to the present embodiment will be described. The optimum miRNA profile is miRNA profile data constituted of optimum miRNA values based on a risk of contracting a disease. An example of the method for generating an optimum miRNA profile will be described below.

First, a miRNA profile of a healthy information provider (healthy person's miRNA profile) is acquired. In order to acquire such a profile, as healthy information providers, mainly young people (people in their 20's and 30's) who have not contracted various diseases and have been confirmed to be at optimum levels in biochemical data relating to blood and various diseases are selected from information providers. It is known that young people generally have a low risk of contracting a disease and that miRNA profiles of young people have a little variation among individuals. A healthy person's miRNA profile is miRNA profile data constituted of a group of each miRNA measurement value of a healthy information provider. For a healthy information provider who has been found not to have contracted various diseases for at least a certain period of time by a follow-up study after blood sampling, only a miRNA profile constituted of miRNA measurement values measured in blood collected in the past by the blood sampling may be used as a healthy person's miRNA profile.

Then, based on the healthy person's miRNA profiles of a plurality of healthy information providers, each of optimum miRNA value is determined. The optimum miRNA values determined in this way are regarded as a group, and miRNA IDs of miRNAs and the optimum miRNA values are associated with each other as shown in FIG. 3 so that an optimum miRNA profile is established.

The optimum miRNA value may be in an optimum range of the miRNA measurement value or may be an optimum miRNA measurement value. The optimum miRNA measurement value may be the mean or median of the miRNA measurement values in an optimum range. Alternatively, the lower limit of the optimum range of the miRNA measurement value of miRNAs promoting a disease may be adopted as an optimum miRNA value, or the upper limit of the optimum range of the miRNA measurement value of miRNAs suppressing a disease may be adopted as an optimum miRNA value. It is preferable that the optimum miRNA profile be a miRNA profile in which a risk of contracting various diseases is the lowest.

### Method for Generating miRNA Importance Table

Next, a method for generating a miRNA importance table according to the present embodiment will be described. The miRNA importance table is generated for each type of disease. The miRNA importance table is tabular data that stores miRNA importance corresponding to each type of disease.

In the present embodiment, for the miRNA increasing the risk of contracting a disease and the miRNA reducing the risk of contracting a disease, an absolute value of the miRNA importance is set as a large value. In the present embodiment, for example, in the case of miRNA increasing the risk of contracting a disease, the miRNA importance is set so that the higher the degree of increasing the risk of contracting a disease (degree of increasing morbidity), the larger the absolute value of the miRNA importance in a “negative direction”. In contrast, in the case of miRNA reducing the risk of contracting a disease, the miRNA importance is set so that the higher the degree of reducing the risk of contracting a disease (degree of reducing morbidity), the larger the absolute value of the miRNA importance in a “positive direction”.

Hereinafter, Examples 1 to 3 of the method for generating a miRNA importance table will be described.

### Example 1 of Method for Generating miRNA Importance Table

FIG. 6 is a flowchart showing Example 1 of the method for generating a miRNA importance table according to the present embodiment. Example 1 of the method for generating a miRNA importance table according to the present embodiment will be described with reference to FIG. 6.

(Step S21) For each type of disease, miRNA disease information showing miRNA suppressing or promoting a disease is acquired. Examples of the miRNA disease information include publicly known documents, such as papers, patent publications, and academic journals, and publicly known information, such as public databases.

(Step S22) Based on the acquired miRNA disease information, miRNA importance corresponding to each type of disease is calculated. For each type of disease, the miRNA importance is calculated based on the degree of increasing morbidity and the degree of reducing morbidity shown in the miRNA disease information. Furthermore, in calculating the miRNA importance based on the degree of increasing morbidity and the degree of reducing morbidity, the number or quality of the miRNA disease information disclosing the degree of increasing morbidity or the degree of reducing morbidity may be added as weighting. Specifically, in a case where the number of pieces of miRNA disease information disclosing the degree of increasing morbidity or the degree of reducing morbidity is large, or in a case where the degree of increasing morbidity or the degree of reducing morbidity is high, a large absolute value is set as the miRNA importance based on the degree of increasing morbidity or the degree of reducing morbidity.

(Step S23) As shown in FIG. 4, the miRNA importance calculated for each type of disease is associated with the miRNA ID of each miRNA and stored in the miRNA importance table for the corresponding type of disease.

### Example 2 of Method for Generating miRNA Importance Table

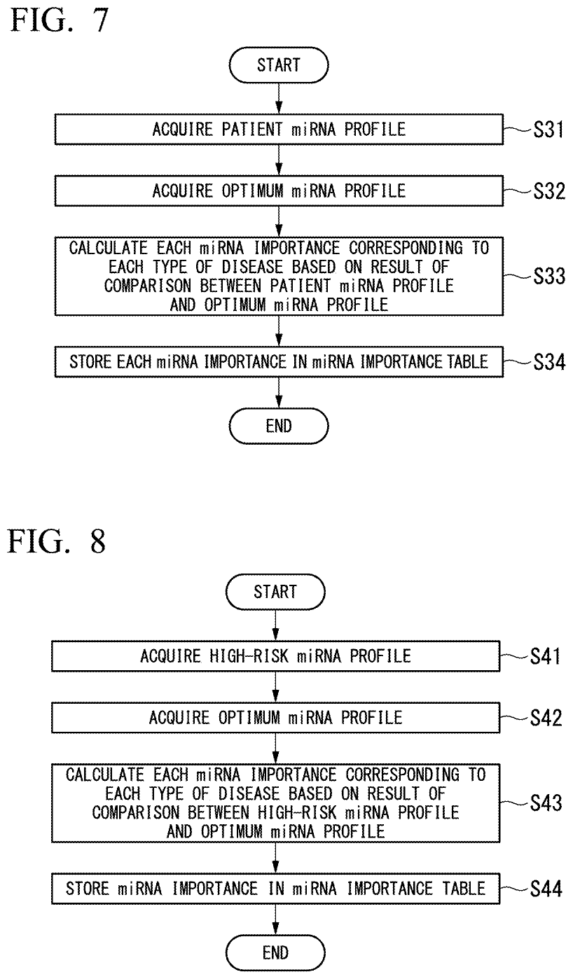

FIG. 7 is a flowchart showing Example 2 of the method for generating a miRNA importance table according to the present embodiment. Example 2 of the method for generating a miRNA importance table according to the present embodiment will be described with reference to FIG. 7.

(Step S31) For each type of disease, a patient miRNA profile constituted of miRNA measurement values of a patient is acquired. The patient is a person suffering from a disease. The patient miRNA profile is miRNA profile data constituted of a group of each miRNA measurement value of the patient.

(Step S32) An optimum miRNA profile is acquired. The optimum miRNA profile is generated by the method for generating an optimum miRNA profile described above.

(Step S33) The patient miRNA profile acquired for each type of disease is compared with the optimum miRNA profile, and miRNA importance corresponding to each type of disease is calculated based on the result of the comparison. For example, for the disease A, miRNA divergence values are calculated from each of the miRNA measurement values in the patient miRNA profile of the disease A and each of the corresponding optimum miRNA values in the optimum miRNA profile. In a case where the miRNA measurement value in the patient miRNA profile of the disease A is higher than the corresponding optimum miRNA value, as miRNA importance in the disease A, a predetermined value showing that the degree of increasing morbidity is high is assigned to the miRNA showing a marked divergence by the calculated miRNA divergence value. In contrast, in a case where the miRNA measurement value in the patient miRNA profile of the disease A is lower than the corresponding optimum miRNA value, as miRNA importance in the disease A, a predetermined value showing that the degree of reducing morbidity is high is assigned to the miRNA showing a marked divergence by the calculated miRNA divergence value.

(Step S34) As shown in FIG. 4, the miRNA importance calculated for each type of disease is associated with the miRNA ID of each miRNA and stored in the miRNA importance table for the corresponding type of disease.

### Example 3 of Method for Generating miRNA Importance Table

FIG. 8 is a flowchart showing Example 3 of the method for generating a miRNA importance table according to the present embodiment. Example 3 of the method for generating a miRNA importance table according to the present embodiment will be described with reference to FIG. 8.

(Step S41) For each type of disease, a high-risk miRNA profile is acquired.

A high-risk miRNA profile is miRNA profile data constituted of a group of miRNA measurement values of an information provider who is at a high risk of contracting a disease but has not yet contracted the disease. The blood used to measure the miRNA measurement values constituting the high-risk miRNA profile may be blood collected from the information provider in the past (for example, cryopreserved blood). Alternatively, the miRNA of the information provider who has not yet contracted a disease may be continuously measured, and the miRNA measurement values obtained from the information provider who has not yet contracted the disease may be used as a high-risk miRNA profile when the information provider contracts the disease.

(Step S42) An optimum miRNA profile is acquired. The optimum miRNA profile is generated by the method for generating an optimum miRNA profile described above.

(Step S43) The high-risk miRNA profile acquired for each type of disease is compared with the optimum miRNA profile, and miRNA importance corresponding to each type of disease is calculated based on the result of the comparison. For example, for the disease A, miRNA divergence values are calculated from each of the miRNA measurement values in the high-risk miRNA profile and each of the corresponding optimum miRNA values in the optimum miRNA profile. In a case where the miRNA measurement value in the high-risk miRNA profile is larger than the corresponding optimum miRNA value, as miRNA importance in the disease A, a predetermined value showing that the degree of increasing morbidity is high is assigned to the miRNA showing a marked divergence by the calculated miRNA divergence value. In contrast, in a case where the miRNA measurement value in the high-risk miRNA profile is smaller than the corresponding optimum miRNA value, as miRNA importance in the disease A, a predetermined value showing that the degree of reducing morbidity is high is assigned to the miRNA showing only a small divergence by the calculated miRNA divergence value.

The time period from when blood used for measuring miRNA of an information provider is collected from the information provider who has not yet contracted a disease to when the information provider contracts the disease (disease-free period) may be added to the miRNA importance in the disease. For example, the shorter the disease-free period, the larger the set absolute value of miRNA importance.

(Step S44) As shown in FIG. 4, the miRNA importance calculated for each type of disease is associated with the miRNA ID of each miRNA and stored in the miRNA importance table for the corresponding type of disease.

Hitherto, Examples 1 to 3 of the method for generating a miRNA importance table have been described.

Each of Examples 1 to 3 of the method for generating a miRNA importance table described above may be used alone, or 2 or more of Examples 1 to 3 may be used in combination. For example, a temporary miRNA importance table is generated by any of Examples 1 to 3 of the method for generating a miRNA importance table, such as Example 1 of the method for generating a miRNA importance table. Then, based on the information of the patient miRNA profile and the optimum miRNA profile in Example 2 of the method for generating a miRNA importance table or the information of the high-risk miRNA profile and the optimum miRNA profile in Example 3 of the method for generating a miRNA importance table, the figures in the temporary miRNA importance table are adjusted. The result of the adjustment is determined as the final miRNA importance table. The method to be used to generate the temporary miRNA importance table may be appropriately selected based on the type of disease and the quantity and quality of information to be obtained by each method.

Furthermore, the method for generating a miRNA importance table may include an information acquisition step of acquiring miRNA attributes, biochemical data, and a miRNA profile of an information provider, in which miRNA importance corresponding to each type of disease may be calculated by additionally using the acquired miRNA attributes, biochemical data, and miRNA profile of the information provider.

The miRNA attributes of the information provider are attributes of the information provider and affect the miRNA measurement values of the information provider. Examples of miRNA attributes include age, gender, race, medical history of the information provider, medical history of relatives of the information provider, and the like. It is preferable that the miRNA attributes include at least age and gender. Only one of the age and gender may be adopted as the miRNA attributes. Note that nationality may be used as information on race.

Examples of the biochemical data include data of the results of biochemical tests on human specimens (such as blood, urine, and stool), images of human skin, ultrasonic images of human beings, X-ray images of human beings, data of the results of a physician's diagnoses on human beings, and the like. The biochemical data of the information provider may be data of test results obtained when the information provider undergoes a medical examination such as health screening.

The miRNA profile of an information provider is miRNA profile data constituted of a group of miRNA measurement values of the information provider.

For example, in order to generate a miRNA importance table for a female-specific disease such as ovarian cancer, miRNA importance is set in consideration of gender. Furthermore, in order to generate a miRNA importance table for a disease having a morbid risk raised by aging, such as dementia, miRNA importance is set in consideration of age.

### Method for Calculating Combined miRNA Score

Next, a method for calculating a combined miRNA score according to the present embodiment will be described.

In the present embodiment, by using a user miRNA profile of a user, the combined miRNA score of the user is calculated for each type of disease. The combined miRNA score of a user for a certain disease, such as the disease A, is the information showing whether or not the miRNA profile is well balanced, in terms of the risk that the user may contract the disease A.

Examples of the combined miRNA score include a miRNA score, a miRNA age, a miRNA deviation, and the like. The miRNA score shows whether or not the miRNA profile is well balanced, by scoring it out of a predetermined perfect score (for example, out of 100). The miRNA age shows whether or not the miRNA profile is well balanced, by using, for example, age.

The miRNA deviation shows whether or not the miRNA profile is well balanced, by using, for example, a value of deviation.

For example, the combined miRNA score is calculated by the following formula. The following formula is a calculation formula for calculating a combined miRNA score of the disease A, in which the user miRNA profile in FIG. 2, the optimum miRNA profile in FIG. 3, and the miRNA importance table in FIG. 4 are used.

Combined miRNA score of disease A=[miRNA divergence value of mirnaid_1 “mirna measurement value_1−optimum mirna value_1”×mirna importance_1A]+[miRNA divergence value of mirnaid_2 “mirna measurement value_2−optimum mirna value_2”×mirna importance_2A]+

In the above formula for calculating the combined miRNA score of the disease A, a difference obtained as a result of subtracting the optimum miRNA value from the miRNA measurement value is calculated as a miRNA divergence value, a product obtained as a result of multiplying the miRNA divergence value by the miRNA importance is calculated as an individual miRNA score, and the sum of individual miRNA scores is calculated as the combined miRNA score.

In the above formula for calculating the combined miRNA score of the disease A, all types of miRNAs may be used for calculation, and the miRNA importance of a miRNA that can be determined as being substantially unrelated to the risk of contracting the disease A may be set to zero. Alternatively, only miRNAs that can be determined as being related to the risk of contracting the disease A may be used for calculation.

For cancer, the combined miRNA score calculation formula to be used may vary with the type of cancer.

In the above formula for calculating the combined miRNA score of the disease A, the difference obtained as a result of subtracting the optimum miRNA value from the miRNA measurement value is calculated as a miRNA divergence value. However, the present invention is not limited thereto. The miRNA divergence value may also be calculated using methods such as performing division between the miRNA measurement value and the optimum miRNA value or performing subtraction or division between the logarithms of the miRNA measurement value and the optimum miRNA value.

In the above formula for calculating the combined miRNA score of the disease A, a product obtained as a result of multiplying the miRNA divergence value by the miRNA importance is calculated as an individual miRNA score. However, the present invention is not limited thereto. The individual miRNA score may be calculated using methods such as performing addition, subtraction, or division between the miRNA divergence value and the miRNA importance, or may be calculated using a quadratic function or a cubic function.

In the above formula for calculating the combined miRNA score of the disease A, the sum of individual miRNA scores is calculated as the combined miRNA score. However, the present invention is not limited thereto. The combined miRNA score may be calculated using methods such as performing subtraction, multiplication, or division on the individual miRNA scores, or may be calculated using a quadratic function or a cubic function.

According to the embodiments described above, by calculating and presenting determination result data such as the combined miRNA score for each type of disease for a user, it is possible to obtain an effect of being able to concisely present the state of balance of the user's miRNA profile for each type of disease. Therefore, it is possible to make the user aware of the risk of contracting the disease A based, for example, on the combined miRNA score of the disease A, and to motivate the user to consider improvement of life habits, such as eating habits, as a measure for improving the balance of the miRNA profile.

In the embodiments described above, the combined miRNA score is calculated for each type of disease. However, morbid risks of a plurality of diseases may be integrated, and an integrated miRNA score may be calculated as an integrated health indicator for the plurality of diseases.

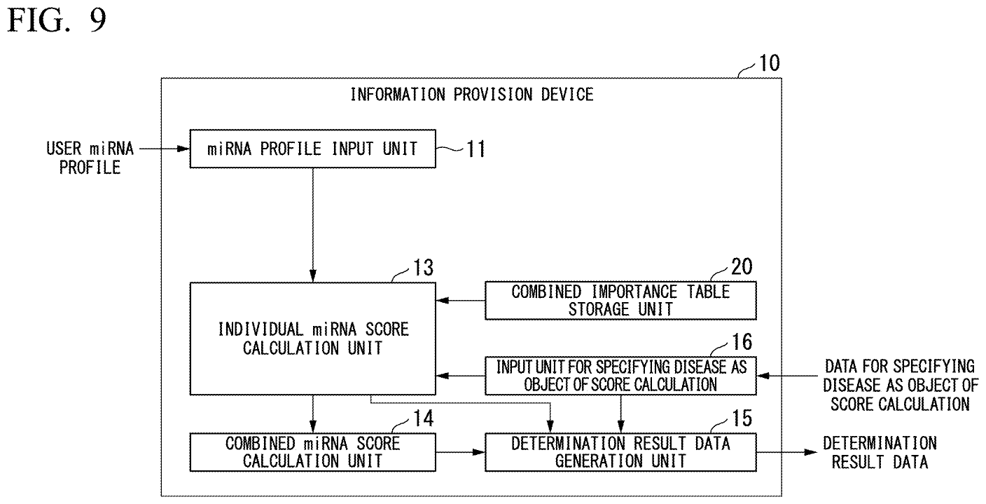

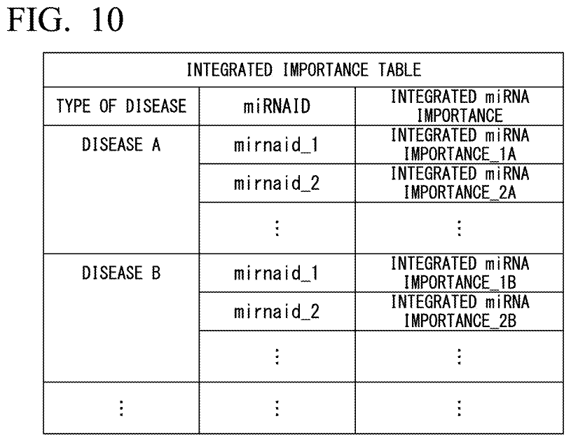

In the embodiments described above, the miRNA divergence value calculation unit 12, the optimum miRNA profile storage unit 17, and the miRNA importance table storage unit 18 are provided. However, the present invention is not limited thereto. For example, an integrated importance table storing integrated miRNA importance values obtained by adding the optimum miRNA value to the miRNA importance may be provided, and the individual miRNA score calculation unit 13 may calculate individual miRNA scores from each of the miRNA measurement values in the user miRNA profile and each of the integrated miRNA importance values in the integrated importance table. FIG. 9 is a block diagram showing an example of a configuration of such an information provision device. The information provision device 10 shown in FIG. 9 includes an integrated importance table storage unit 20 that stores an integrated importance table. As shown in FIG. 10, for each type of disease, the integrated importance table stores integrated miRNA importance values which are determined by adding miRNA importance corresponding to each type of disease to an optimum miRNA value based on risk of contracting a disease. In the information provision device 10 shown in FIG. 9, the individual miRNA score calculation unit 13 calculates individual miRNA scores from each of the miRNA measurement values in the user miRNA profile and each of the integrated miRNA importance values in a disease as an object of score calculation in the integrated importance table. The determination result data generation unit 15 generates and outputs determination result data based on the individual miRNA scores.

Furthermore, a computer program for realizing the function of the information provision device described above may be recorded on a computer-readable recording medium, and the program recorded on the recording medium may be read by the computer system and executed. “Computer system” mentioned herein may include hardware such as OS and peripheral devices.

In addition, “computer-readable recording medium” refers to a storage device like a writable non-volatile memory such as a flexible disk, a magneto-optical disk, ROM, or a flash memory, a portable medium such as a DVD (Digital Versatile Disc), or a built-in hard disk in a computer system.

Furthermore, “computer-readable recording medium” also includes a recording medium that holds a program for a certain period of time, such as a volatile memory (for example, DRAM (Dynamic Random-Access Memory)) in a computer system that functions as a server or client in a case where a program is transmitted through a network such as the Internet or a communication channel such as a telephone circuit.

The aforementioned program may also be transmitted from a computer system having a storage device or the like storing the program to another computer system, through a transmission medium or by a transmission wave in the transmission medium. “Transmission medium” for transmitting a program refers to a medium having a function of transmitting information, such as a network (communications network) like the Internet or a communication channel (communication line) like a telephone circuit.

The aforementioned program may be used for realizing some of the functions described above.

In addition, the program may also be a so-called difference file (difference program), which can realize the above functions in combination with a program pre-recorded on a computer system.

Hitherto, the present invention has been described using embodiments. It goes without saying that the technical scope of the present invention is not limited to the scope described in the above embodiments. It is apparent to those skilled in the art that various changes or improvements can be added to the above embodiments. Furthermore, as is evident from the description of Claims, the embodiments obtained by adding such changes or improvements can also be included in the technical scope of the present invention.

### REFERENCE SIGNS LIST

10: information provision device
11: miRNA profile input unit
12: miRNA divergence value calculation unit
13: individual miRNA score calculation unit
14: combined miRNA score calculation unit
15: determination result data generation unit
16: input unit for specifying a disease as an object of score calculation
17: optimum miRNA profile storage unit
18: miRNA importance table storage unit
20: integrated importance table storage unit

1.-8. (canceled)

9. A device for determining a risk of contracting multiple types of cancers, comprising:
a miRNA profile input unit for receiving a user miRNA profile that includes miRNA measurement values of the user;
an individual miRNA score calculation unit for calculating individual miRNA scores based on each of the miRNA measurement values in the user miRNA profile, each of the individual miRNA scores corresponding to a risk of contracting a disease; and
a determination result data generation unit for generating and outputting determination result data based on the individual miRNA scores.

10. The device according to claim 9, further comprising:
an optimum miRNA profile storage unit for storing an optimum miRNA profile that includes optimum miRNA values based on a risk of contracting a disease;
a miRNA divergence value calculation unit for calculating a miRNA divergence value from each of the miRNA measurement values in the user miRNA profile and each of the corresponding optimum miRNA values in the optimum miRNA profile, the miRNA divergence value showing a divergence between a respective miRNA measurement value and a corresponding optimum miRNA value; and
a miRNA importance table storage unit for storing, by a type of cancer of the multiple types of cancers, a miRNA importance table that includes miRNA importance values each showing a degree of importance of each of miRNAs to a risk of contracting a type of cancer in a period when the user is not yet contracted a disease,
wherein the individual miRNA score calculation unit calculates individual miRNA scores from each of the miRNA importance values of a type of cancer as an object of score calculation listed in the miRNA importance table and each of the miRNA divergence values.

11. The device according to claim 10, further comprising:
a high-risk miRNA profile acquisition unit for acquiring a high-risk miRNA profile for each of the multiple types of cancers, the high-risk miRNA profile including miRNA measurement values of an information provider who is above a certain level of risk of contracting a type of cancer but has not yet contracted the type of cancer;
an optimum miRNA profile acquisition unit for acquiring an optimum miRNA profile constituted of optimum miRNA values based on a risk of contracting a disease;
a miRNA importance calculation unit for comparing the high-risk miRNA profile for each of the multiple types of cancers acquired by the high-risk miRNA profile acquisition unit with the optimum miRNA profile acquired by the optimum miRNA profile acquisition unit, and calculates miRNA importance corresponding to each of types of cancers based on a result of the comparison; and
a miRNA importance table generation unit for storing, by a type of cancer of the multiple types of cancers, the miRNA importance corresponding to each of types of cancers calculated by the miRNA importance calculation unit in a miRNA importance table.

12. The device according to claim 9, further comprising:
an integrated importance table storage unit for storing, by a type of cancer of the multiple types of cancers, an integrated importance table that includes integrated miRNA importance values for each type of disease, each of the integrated miRNA importance values is determined by adding a miRNA importance showing degree of importance of each of miRNAs to a risk of contracting each of types of cancer in a period when the user is not yet contracted a disease to each optimum miRNA value based on a risk of contracting a disease;
wherein the individual miRNA score calculation unit that calculates individual miRNA scores from each of the miRNA measurement values in the user miRNA profile and each of the integrated miRNA importance values of a type of cancer as an object of score calculation listed in the integrated importance table.

13. The device according to claim 9, further comprising:
a miRNA score calculation unit for calculating a miRNA score by combining the individual miRNA scores.

14. The device according to claim 9, further comprising:
an input unit for specifying a disease as an object of score calculation that receives an input of data for specifying a type of cancer as an object of score calculation showing a disease as an object of score calculation.

15. A method for providing information about risk of contracting multiple types of cancers, comprising:
receiving a user miRNA profile constituted of miRNA measurement values of a user;
storing an optimum miRNA profile constituted of optimum miRNA values based on a risk of contracting a disease;
calculating a respective miRNA divergence value from each of the miRNA measurement values and each corresponding optimum miRNA value in the optimum miRNA profile, wherein the respective miRNA divergence value shows divergence between the miRNA measurement value and the corresponding optimum miRNA value;
storing a miRNA importance table that includes miRNA importance values showing a degree of importance of each type of miRNA to a risk of contracting a type of cancer in a period when the user is not yet contracted cancer;
calculating individual miRNA scores from each of the miRNA importance values of a type of cancer as an object of score calculation listed in the miRNA importance table and each of the miRNA divergence values; and
generating and displaying the individual miRNA scores.

16. The method according to claim 15, further comprising:
acquiring miRNA disease information showing miRNA suppressing or promoting a cancer for each type of cancer;
calculating second miRNA importance values corresponding to each type of cancer based on the miRNA disease information; and
storing each of the second miRNA importance values in the miRNA importance table.

17. The method according to claim 15, further comprising:
acquiring a patient miRNA profile that includes miRNA measurement values of a patient for each type of cancer;
acquiring an optimum miRNA profile that includes optimum miRNA values based on a risk of contracting a disease;
comparing the patient miRNA profile acquired for each type of cancer with the optimum miRNA profile; and
calculating second miRNA importance values corresponding to each type of cancer based on results of the comparison; and
storing each of the second miRNA importance values in the miRNA importance table.

18. The method according to claim 15, further comprising:
acquiring a high-risk miRNA profile for each type of cancer, the high-risk miRNA profile including miRNA measurement values of an information provider who is above a certain level of risk of contracting a cancer but has not yet contracted the cancer;
acquiring an optimum miRNA profile constituted of optimum miRNA values based on a risk of contracting a disease;
comparing the high-risk miRNA profile acquired for each type of cancer with the optimum miRNA profile;
calculating second miRNA importance values corresponding to each type of cancer based on the comparing; and
storing each of the second miRNA importance values calculated for each type of cancer in the miRNA importance table.

19. The method according to claim 18, further comprising:
acquiring miRNA attributes, biochemical data, and a miRNA profile of the information provider; and
calculating the miRNA importances corresponding to each type of cancer using the acquired miRNA attributes, biochemical data, and miRNA profile of the information provider.
